(19) United States
(12) Patent Application Publication
(10) Pub. No.: US 2011/0071613 A1
(43) Pub. Date: Mar. 24, 2011

(54) INTEGRATED STENT RETRIEVAL LOOP ADAPTED FOR SNARE REMOVAL AND/OR OPTIMIZED PURSE STRINGING

(75) Inventors: Mark Wood, Shrewsbury, MA (US); Devon Amos, Boston, MA (US); Paul K. Norton, Lunenburg, MA (US)

(73) Assignee: BOSTON SCIENTIFIC SCIMED, INC., Maple Grove, MN (US)

(21) Appl. No.: 12/878,125

(22) Filed: Sep. 9, 2010

Related U.S. Application Data
(60) Provisional application No. 61/244,206, filed on Sep. 21, 2009.

(51) Int. Cl. A61F 2/84; A61F 2/82; B21D 35/00

(52) U.S. Cl. 623/1.11; 623/1.15; 72/362

(57) ABSTRACT

The present invention includes a braided stent and method of making the same. The braided stent has an integral retrieval and/or repositioning member. The stent includes a first open end, a second open end and a tubular body therebetween. The retrieval and/or repositioning member extends from and is interbraided into the braided tubular body. The retrieval and/or repositioning member includes an elongated portion extending from the first open end and a second section interlooping circumferentially about the first open end such that force exerted on the elongated portion causes radially contraction of the stent end and stent body.

### CROSS-REFERENCE TO RELATED APPLICATION

This application claims the benefit of U.S. Provisional Application No. 61/244,206, filed Sep. 21, 2009, the contents of all of which are incorporated by reference herein.

### FIELD OF THE INVENTION

The present invention relates to devices, methods and systems for retrieval and/or repositioning of an implanted stent. More particularly, the present invention relates to implantable stents having a stent retrieval member or loop for easy retrieval and/or repositioning of the implanted stent.

### BACKGROUND OF THE INVENTION

An intraluminal prosthesis is a medical device used in the treatment of diseased bodily lumens. One type of intraluminal prosthesis used in the repair and/or treatment of diseases in various body vessels is a stent. A stent is a generally longitudinal tubular device formed of biocompatible material which is useful to open and support various lumens in the body. For example, stents may be used in the vascular system, urogenital tract, esophageal tract, tracheal/bronchial tubes and bile duct, as well as in a variety of other applications in the body. These devices are implanted within the vessel to open and/or reinforce collapsing or partially occluded sections of the lumen.

Stents generally include an open flexible configuration. This configuration allows the stent to be inserted through curved vessels. Furthermore, this configuration allows the stent to be configured in a radially compressed state for intraluminal catheter implantation. Once properly positioned adjacent the damaged vessel, the stent is radially expanded so as to support and reinforce the vessel. Radial expansion of the stent may be accomplished by inflation of a balloon attached to the catheter or the stent may be of the self-expanding variety which will radially expand once deployed.

Prior retrieval systems, for example as described in U.S. Pat. No. 6,821,291 to Bolea et al., may appear easy to use, but often are limited to a specific tool for removal and/or require certain user-sensitive techniques, such as twisting or turning in order to reposition or remove the stent. Moreover, in smaller stents, such as biliary stents, the spacing between conventional stent segments is generally smaller than the size of standard forceps or graspers, making it even difficult to grab a portion of the stent.

### SUMMARY OF THE INVENTION

The present invention provides an implantable device, for example a stent, including a braided stent, having a retrieval and/or repositioning member. The implantable device is formed from one or more elongated filaments wound or braided to form a tubular device having opposed first open end, a second open end, a tubular body therebetween. The device has an interior surface and an exterior surface. The retrieval and/or repositioning member includes an elongated portion extending from the first open end and the retrieval and/or repositioning member interlooping circumferentially about the first open end such that force exerted on the elongated portion causes radially contraction of the device.

In another aspect of the present invention, one or more elongated filaments are wound or braided to form a tubular implantable device or stent having a retrieval and/or repositioning member and opposed first open end and a second open end with each open end having a circumference and a tubular body therebetween is provided. The first open end is defined by series of closed-end loops. The retrieval and/or repositioning member has a first section including at least one elongated closed-end loop extending from the first open end, and a second section emerging from the braided tubular body, interwoven with at least one closed-end loop and integrally extending into the first section whereby force exerted on the elongated closed-end loop causes radially contraction of the tubular device.

In a further aspect of the present invention, a method for producing a tubular wound or braided implantable device or stent having opposed first end and second end and having an integral retrieval and/or repositioning loop at the first end is provided. The tubular wound or braided device or stent includes the steps of selecting a single elongate biocompatible filament having opposed ends; forming a retrieval and/or repositioning member from the single filament comprising an elongated loop which extends above the first end to permit grabbing of the loop by a practitioner to radially contract the stent; and winding or braiding the single filament, optionally with other filaments, to form the device or stent.

### BRIEF DESCRIPTION OF THE DRAWINGS

FIG. 1 is a partial expanded view of a stent of the prior art.

FIG. 2 is another view of the prior art stent of FIG. 1 being pulled by a retrieval device.

FIG. 3 depicts a braided stent with closed-end loop design having a retrieval and/or repositioning member of the present invention.

FIG. 4 is a perspective view of one end of the stent of FIG. 3 having a retrieval and/or repositioning member according to the present invention.

FIG. 5 is an expanded view of the retrieval and/or repositioning member of the present invention.

FIG. 6 is an expanded view of the retrieval and/or repositioning member of the present invention being pulled by a retrieval device.

FIG. 7 is another view of the retrieval and/or repositioning member of FIG. 6 in a retracted or compressed state.

FIG. 8 is an expanded view of the stent end of the present invention including a retrieval and/or repositioning member having an outwardly directed bent portion.

FIG. 9 is a side view of the stent end of FIG. 8.

FIG. 10 is an expanded view of the stent end of the present invention including a retrieval and/or repositioning member having an inwardly directed bent portion.

FIG. 11 is an expanded view of the stent end of the present invention including a retrieval and/or repositioning member having an outwardly directed twisted portion.

FIG. 12 is an expanded view of the stent end of the present invention including a retrieval and/or repositioning member having an inwardly directed twisted portion.

### DETAILED DESCRIPTION

FIG. 1 depicts a prior art stent 10 including a retrieval and/or repositioning loop 12. The retrieval and/or repositioning loop 12 includes two wires 14, 18 that are circumferentially disposed about the end 16 of stent 10. The two wires 14, 18 extend outwardly from the stent end 16 to permit access to the stent 10 by a practitioner with a retrieval device such as rat tooth forceps or hooking device. The two wires 14, 18 cooperatively work in conjunction with each other to cinch the end 16 of the stent 10 and radially contract the stent 10 body when pulled on by a retrieval device. Specifically, when the two wires 14, 18 of the retrieval and/or repositioning loop 12 are accessed and pulled, the two wires 14, 18 slide by each other to allow the stent end 16 to cinch. Once the stent end 16 is cinched, then continued pulling on the retrieval and/or repositioning loop 12 axially compresses or radially contracts the body of the stent 10 from the cinched stent end down to the other end. In use, however, both wires 14, 18 are pulled together to provide for radial contraction of the stent 10. Further, these wires 14, 18 slide about each other when being pulled to permit radial contraction of the stent 10. The retrieval and/or repositioning loop 12 of the prior art may use a retrieval device such as rat tooth forceps or hook to engage its retrieval and/or repositioning loop because the other devices may pinch the two wires 14, 18 and thereby hindering the two wires 14, 18 from sliding by each other. Other useful retrieval devices include but are not limited to needle nose pliers, radial jaws or a snare. Further details of this prior art stent 10 with its retrieval and/or repositioning loop 12 may be found in U.S. Patent Application Publication No. 2006/0276887 to Brady et al., the contents of which are incorporated herein by reference. Moreover, a retrieval and/or repositioning loop which is not integrally from the wires braided to form a stent is disclosed in U.S. Patent Application Publication No. 2006/0190075 to Jordan et al., the contents of which are incorporated herein by reference.

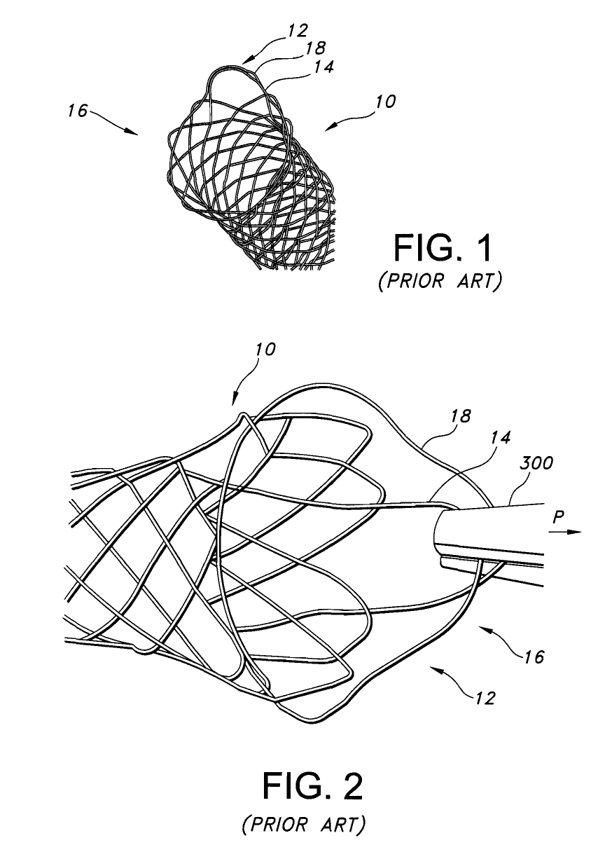

FIG. 2 shows the prior art stent 10 being pulled (P) by a retrieval device 300 in the direction away from the stent end 16. The retrieval device 300 hold both wires 14, 18 in a fixed position as it pulls the stent 10. The retrieval device 300 hinders the wires 14, 18 from moving about each other which causes the stent end 16 to remain in a flared state and prevent the wires 14, 18 from fully cinching the stent end 16. Additionally, FIG. 2 shows that the stent 10 body is not fully radially contracted due to the reduced ability of the wires 14, 18 to slide about each other.

Thus, there is a need for a single wire retrieval and/or repositioning member that provides both improved stent end cinching and improved stent body radial contraction. Further, there is a need for a single wire retrieval and/or repositioning member that is capable of cinching the end of the stent and radially contracting the stent body using a variety of devices used by a practitioner. Furthermore, there is a need for a single wire retrieval and/or repositioning member that provides for substantially even radial contraction of the stent end and stent body and permits easy access by a practitioner to the pulling member of the stent.

The present invention provides at least one single wire retrieval and/or repositioning member which is integral and formed from one of the filaments or wires used to form the braided stent. The retrieval and/or repositioning member is designed to provide a structure which has the required tensile strength to prevent fracture or damage to the stent when force is applied to reposition or retrieve the stent, yet allows for a very low delivery profile such that it can easily be loaded onto a delivery device without interfering with the deployment into the body or requiring increased deployment force. Because the retrieval and/or repositioning member is an integral part of the actual braiding or winding for the stent structure per se, as opposed to being a separate add-on element, no joining, i.e., welding, crimping, twisting, of the retrieval and/or repositioning member to the stent structure is necessary. Tensile strength of the retrieval and/or repositioning member may thus be maximized while concomitantly maintaining the lowest profile for delivery to a patient. The wire or wires used to form at least one retrieval and/or repositioning member may be of the same type and material as the other wires forming the braided stent, or alternatively they may be made from different types or materials. In one desirable embodiment, the retrieval and/or repositioning member is made from a single wire which is also used to form the braided stent or at least part of the braided stent. In this manner, the retrieval and/or repositioning member can further seamlessly transition into the body of the stent. As used herein, the phrase “retrieval and/or repositioning member” refers to a retrieval member, a repositioning member, or a combination thereof which is integrally formed with a stent and, when a longitudinally pulling force is applied thereto, aids in the radial contraction or cinching of the entire stent equally to facilitate movement, retrieval and/or repositioning of the stent.

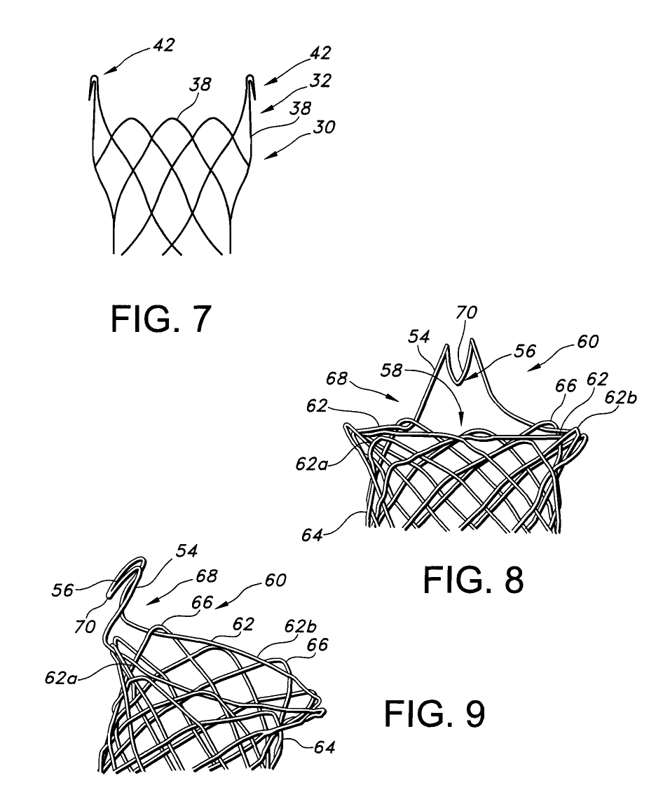

More than one retrieval and/or repositioning member may be incorporated into the stent. For example, each stent end might have one or more retrieval and/or repositioning members, as shown in FIG. 7. In some embodiments only one retrieval and/or repositioning member is present at one or more ends.

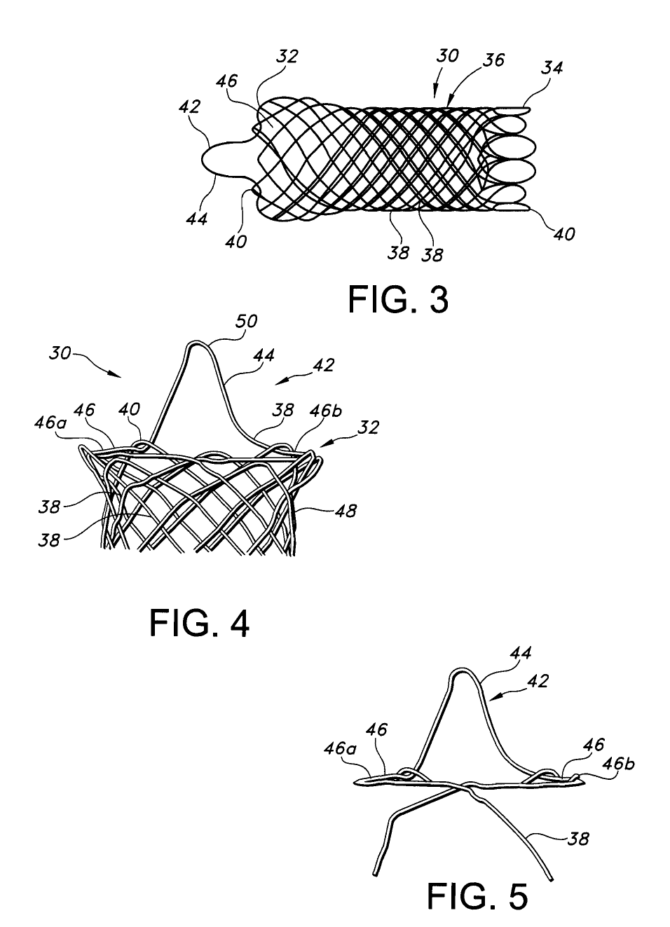

FIG. 3 depicts stent 30 of the present invention. Stent 30 is a hollow tubular structure having opposed first and second open ends 32, 34 and having a tubular wall 36 therebetween. The tubular wall 36 has a plurality of elongate wires 38 formed into the tubular wall 36. The elongate wires 38 traverse the length of the stent 30 in a direction traverse to the longitudinal length of the stent 30. The elongate wires 38 may be formed into the tubular wall 36 by braiding the wires 38, winding the wires 38 or even winding a single wire 38, knitting the wires 38, and combinations thereof. Preferably, the wires 38 are braided in a braided pattern to form the tubular wall 36. A useful nonlimiting braided pattern includes a one over and one under pattern, but other patterns may suitably be used.

As depicted in FIG. 3, stent 30 is desirably an atraumatic stent having close-loop ends 40 defining the circumference of the opposed first and second open ends 32, 34. The elongate wire 38 terminating at open end 32 are looped over to form a closed-end loop 40 and reintroduced into the stent to form the completed braided sent. After the braided stent is formed, the ends of the wire may be welded or otherwise connected together forming a braided stent with no open ends, open loops or sharp edges. Additionally, multiple wires 38 may be used in forming the stent and the wires 38 are mated to form closed-loops and adjacently mated wires are secured to one and the other by mechanical means, such as welds.

The stent 30 is desirably an atraumatic stent having no sharp terminating members at one or both of the opposed first and second open ends 32, 34. The elongate wires 38 terminating at open end 32 are mated to form closed-end loops 40 and adjacently mated wires are secured to one and the other by mechanical means, such as welds. The positioning of adjacently mated wires to form closed-end loop designs is further described in U.S. Published Application Nos. US 2005-0049682 A1, and 2006-0116752 A1, the contents of all which are incorporated herein by reference. Desirably, the elongate wires 38 terminating at open end 32 are in a cathedral type arch or loop configuration. Further details of the cathedral type of arch or closed-loop configuration may be found in U.S. Application Publication No. 2005-0256563 A1, the contents of which are incorporated herein by reference. In any event, the current invention is useful with various stent designs, including those without atraumatic ends.

Desirably, the wires 38 are made from any suitable implantable material, including without limitation nitinol, stainless steel, cobalt-based alloy such as Elgiloy®, platinum, gold, titanium, tantalum, niobium, polymeric materials and combinations thereof. Useful and nonlimiting examples of polymeric stent materials include poly(L-lactide) (PLLA), poly(D,L-lactide) (PLA), poly(glycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D,L-lactide-co-caprolactone) PLA/PCL), poly(glycolide-co-caprolactone) (PGA/PCL), polyphosphate ester) and the like. Wires made from polymeric materials may be also include radiopaque materials, such as metallic-based powders, particulates or pastes which may be incorporated into the polymeric material. For example the radiopaque material may be blended with the polymer composition from which the polymeric wire is formed, and subsequently fashioned into the stent as described herein. Alternatively, the radiopaque material may be applied to the surface of the metal or polymer stent. In either embodiment, various radiopaque materials and their salts and derivatives may be used including, without limitation, bismuth, barium and its salts such as barium sulphate, tantulaum, tungsten, gold, platinum and titanium, to name a few. Additional useful radiopaque materials may be found in U.S. Pat. No. 6,626,936, which is herein incorporated in its entirely by reference. Metallic complexes useful as radiopaque materials are also contemplated. The stent may be selectively made radiopaque at desired areas along the wire or made be fully radiopaque, depending on the desired end-product and application. Further, the wires 38 have an inner core of tantalum, gold, platinum, iridium or combination of thereof and an outer member or layer of nitinol to provide a composite wire for improved radiocapicity or visibility. Desirably, the inner core is platinum and the outer layer is nitinol. More desirably, the inner core of platinum represents about at least 10% of the wire based on the overall cross-sectional percentage. Moreover, nitinol that has not been treated for shape memory such as by heating, shaping and cooling the nitinol at its martensitic and austenitic phases, is also useful as the outer layer. Further details of such composite wires may be found in U.S. Patent Application Publication 2002/0035396 A1, the contents of which is incorporated herein by reference. Preferably, the wires 38 are made from nitinol, or a composite wire having a central core of platinum and an outer layer of nitinol. Further, the filling weld material, if required by welding processes such as MIG, may also be made from nitinol, stainless steel, cobalt-based alloy such as Elgiloy, platinum, gold, titanium, tantalum, niobium, and combinations thereof, preferably nitinol. The material of the cathode is no critical and can be made out of any suitable metal. The filling weld material and the wire 38 may be made of the same material, for example nitinol.

Further, the wires 38 may have a composite construction, such as described found in U.S. Patent Application Publication 2002/0035396 A1, the contents of which is incorporated herein by reference. For example, the wires 38 may have an inner core of tantalum gold, platinum, iridium or combination of thereof and an outer member or layer of nitinol to provide a composite wire for improved radiocapicity or visibility. Preferably, the wires 38 are made from nitinol.

Either or both of the opposed open ends 32, 34 of the stent 30 may have a retrieval and/or repositioning member 42 thereat. The retrieval and/or repositioning member 42 is useful for retrieval and/or repositioning of an implanted or deployed stent 30. The retrieval and/or repositioning member 42 allows a practitioner to contract and move, reposition and/or retrieve the stent 30 within an implanted lumen (not shown). The retrieval and/or repositioning member 42 may be made from a wire, including but not limited to a memory shape alloy, such as the above described materials, including nitinol. The use of a wire, as compared other convention materials such as suture thread, has numerous advantages. For example, the self-supporting nature of the wire facilitates the locating of the retrieval and/or repositioning member. A wire will not tangle, a potential problem with suture loops, especially with suture loops made from natural or polymeric threads or filaments, and will also aid in opening the stent 30. Another advantage from using a wire material is the wire loop defining the retrieval and/or repositioning member would be less likely to break than a plastic or polymeric loop when a pulling force is applied, such as required for repositioning or removal of the stent 30.

As depicted in FIGS. 4-5 the stent 30 includes the retrieval and/or repositioning member 42. The retrieval and/or repositioning member 42 includes a first section 44 and a second section 46. The retrieval and/or repositioning member 42 may be angularly bent and extends away from the stent end 34 defining the first section 44. The first section 44 extends upwardly away from the stent end 34 forming a loop, arc or inverted “U” shaped geometry. The retrieval and/or repositioning member 42 may include a second section 46 that extends from the angularly bent portion or either ends of the first section 44. The second section 46 may only traverse partially about the circumference of the stent end 34. The second section 46 may include two legs 46a, 46b that emerge from the braid 48 of the stent 30 and cross over or under each other prior to traversing the circumference to connect to either end of the first section 44. In other words, the legs 46a, 46b are contained within, and a formation of, the braided pattern 48 of the stent 30. One or more continuous wire(s) 38 is used to form stent 30. Generally, wire 38 is bent to form the arc of the first section 44. Wire 38 on either side of the first section 44 can be angularly bent such that the first section 44 will extend away from the stent end. Each leg 46a, 46b of the second section 46 can extend perpendicularly from the first section 44. Each leg 46a, 46b can be formed into a half circle. Together the two half circles, legs 46a, 46b, make up the second section 46 of the retrieval and/or repositioning member 42 and define the circumference of the stent end 34. Then, the second section 46 enters into the normal braiding pattern 48 of the stent 30 and the stent body is formed using a braided pattern. The retrieval and/or repositioning member 42 may be angularly bent and extend away from the stent end 34 defining the first section 44 thereby having a length that partially circumvents the circumference of the stent end 34. The first section 44 can be provided with a circumferential length to permit easy access by a practitioner of the retrieval and/or repositioning member 42.

As depicted in FIG. 4, the retrieval and/or repositioning member 42 may pass through a closed-end loop 40 to attach the elongated portion or first section 44 to the stent end 32 to facilitate cinching of the stent end 32 when pulling force is applied thereto. The wire 38 forming the second section 46 of the retrieval and/or repositioning member 42 may cross through some or all of the closed-end loops 40 at stent end 32. Some of the closed-end loops 40 may be longitudinally offset from other of the closed-end loops 40, and the wire 38 may suitably cross through those closed-end loops 40 at the very end of the stent 30 while not crossing through the closed-end loops 40 that are disposed inwardly from the stent end 32. Alternatively, the stent 30 may have no offsetting of the closed-end loops 40 at stent end 34. It is desirable that the second section 46 of the retrieval and/or repositioning member 42 passes through at least two closed-end loops 40. For example, each leg 46a, 46b may pass through at least one closed-end loop 40 to attach each side of the elongated first section 44 to the stent end 32 to facilitate cinching of the stent end 32 when pulling force is applied thereto.

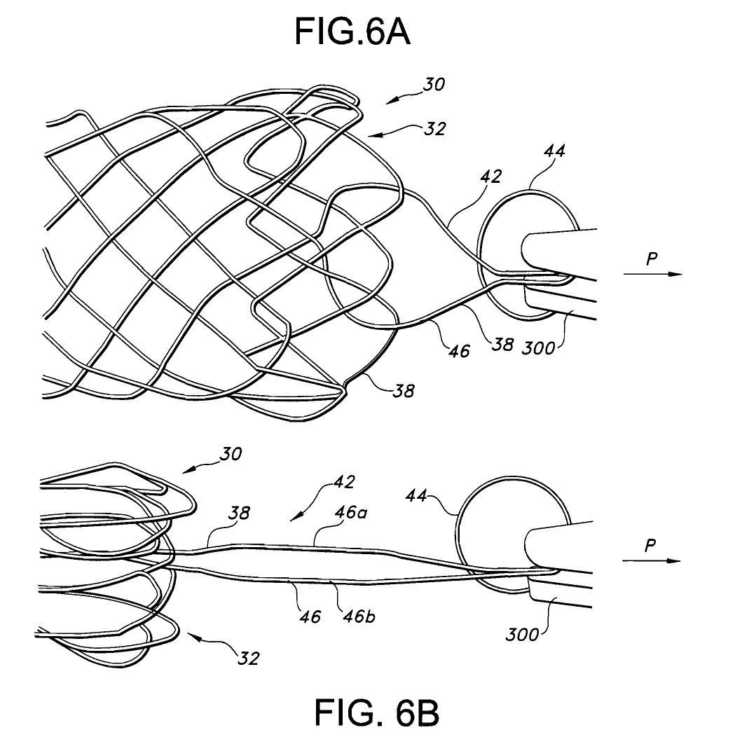

As depicted in FIGS. 6a-6b, the stent 30 should easily contract upon application of a pulling force, “1”, to the retrieval and/or repositioning member 42. The first section 44 of the retrieval and/or repositioning member 42 is accessed and pulled by a tool of a physician, such as by apex or loop 50 of the retrieval and/or repositioning member 42. The legs 46a, 46b of the second section 46 are pulled away from the stent end 34 by the pull force (P). The stent end 34 is axially compressed or radially contracted by a cinching action of the circumferential portion of the wire 38. At the same time the legs 46a, 46b are pulled away from the stent end 34, the legs 46a, 46b pull on the braided wire 38 extending through the braided stent body. Wire 38 is pulled toward and away from the stent end 34 causing longitudinal contraction substantially equally along the length of the stent 30. Further, as the wire 38 forming both the first and second sections 44, 46 is integral with the braid pattern 48 of the stent 30, such integral wire further facilitates movement, repositioning or retrieval of the stent 30 by, among other things, providing a cinching or radially contracting action substantially equally along the longitudinal length of the stent and also by transferring the pulling force along the longitudinal length of the stent. Thus, the pulling of the retrieval and/or repositioning member 42 provides for simultaneous contracting and pulling of the stent 30. In contrast, if a pulling force (P) is applied to an end of a stent without having a retrieval and/or repositioning member 42, there is no cinching or radial contracting force generated at that stent end.

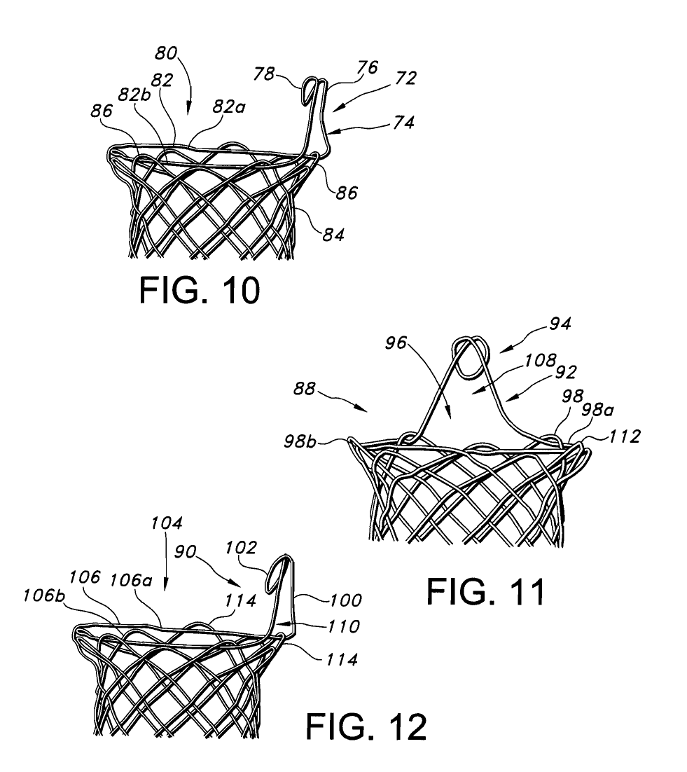

As depicted in FIG. 4, the retrieval and/or repositioning member 42 desirably extends longitudinally outward from the braided stent body and about the circumference of the stent end 32 forming the second section 46 and extending outwardly away from the circumference forming a first section of the stent end 34. Such extended and elongated retrieval and/or repositioning member 42 facilitates grabbing of the retrieval and/or repositioning member 42 by a practitioner. The first section of the retrieval and/or repositioning member may be formed in various geometric shapes to allow for ease of access by the practitioner and allow for equal longitudinal contraction of the stent. FIGS. 4-5 depict the first section 44 having a loop geometry 50. FIGS. 8-10 depict a loop 50 bent downwardly over itself. Specifically, FIG. 10 depicts a first section 76 with a bent portion 78 bent downwardly over itself and bent towards the center of the stent lumen. FIGS. 8-9 depict a bent portion 56 bent downwardly towards itself and bent outwardly away from the center of the stent lumen. FIGS. 11-12 depict loops twisted into a closed-loop or pretzel shaped geometry. Specifically, FIG. 11 depicts a first section 92 twisted and bent towards the center of the stent lumen. FIG. 12 depicts a first section 100 twisted and bent outwardly away from the center of the stent lumen. The various configurations of the retrieval and/or repositioning members will be described in further detail below.

In further detail, stent 60 of FIGS. 8-9 is similar to stent 30 of FIGS. 4-5 including a braided stent body and retrieval and/or repositioning member. The retrieval and/or repositioning member 68 may include a first section 54 angularly bent and extending above the stent end; and a second section 62 extending perpendicularly from the first section and merging into and being integrally part of the formation of the braided stent body. In contrast to stent 30, FIGS. 8-9 depict the first section 54 having a bent portion 56 instead of loop 50 of the FIGS. 4-5. The bent portion 56 forms a loop 70, similar to loop 50, bent downwardly toward the stent end. The bent portion 56 may be directed towards the exterior of the stent and outwardly away from the center lumen 58 of the stent 60. FIG. 9 depicts a side view of the bent portion 56 showing the inverted “J” shape or hook shape of the bent portion 56. FIG. 8 depicts the front view of the second section 62 showing two legs 62a, 62b that emerge from the braided stent body 64 and cross over, or under, each other prior to traversing the circumference to connect to either end of the first section 54. FIG. 8 shows legs 62a, 62b interconnected or woven into a couple of closed-end loops 66. The stent 60 advantageously permits a practitioner to grab the bent portion 56 across the hook formed by the bent portion 56.

FIG. 10 depicts stent 72 which is similar to stent 60 of FIGS. 8-9 including a braided stent body and a retrieval and/or repositioning member. The retrieval and/or repositioning member may include a first section extending above the stent end and including a bent portion and a second section emerging from and integrally formed from the braided stent body. In contrast to stent 60, FIG. 10 depicts the first section 76 having a bent portion 78 bent downwardly toward the stent end and inwardly toward the center lumen 80 of the stent 72. FIG. 10 depicts a back view of the bent portion 76 showing the inverted “J” shape or hook shape of the bent shaped member. The second section 82 may include two legs 82a, 82b that emerge from the braided stent body 84 and cross over, or under, each other prior to traversing the circumference to connect to either end of the first section 76. Legs 82a, 82b may be interconnected or woven into a couple of closed-end loops 76. The stent 72 advantageously permits a practitioner to grab the bent portion 78 across the inwardly facing hook formed by the bent portion 78.

FIGS. 11 and 12 depict stents 88 and 90, respectively, which are similar to stents 60 and 72 of FIGS. 9 and 10, respectively, including a braided stent body and a retrieval and/or repositioning member. The retrieval and/or repositioning member includes a first section extending above the stent end and including a bent portion, and a second section emerging from and integrally formed from the braided stent body. In contrast to stents 60 and 72, FIGS. 11 and 12 depict the first sections 92, 100, respectively, having a bent portion which is twisted into a closed-loop, for example a pretzel shaped, geometry defining a twisted portion. FIG. 11 depicts a first section 92 including an outwardly directed twisted portion 94 which is a loop that is bent downwardly towards the stent end, directed outwardly away from the center lumen 96 and twisted in a closed-loop or pretzel formation. FIG. 11 depicts a front view of the twisted portion 94 showing the twisted inverted loop formed into a pretzel shaped geometry. The second section 98 can include two legs 98a, 98b that emerge from the braided stent body and cross over, or under, each other prior to traversing the circumference to connect to either end of the first section 92. Legs 98a, 98b can be interconnected or woven into two or more closed-end loops 112. Stent 90 of FIG. 12 is similar to stent 88 of FIG. 11. In contrast to stent 88, stent 90 may include first section 100 including an inwardly twisted portion 102 which is a loop bent downwardly towards the stent end, directed inwardly towards the center lumen 104 and twisted in a closed-loop or pretzel formation. FIG. 12 depicts a back-side view of the twisted portion 102 showing the twisted inverted loop formed into a pretzel shaped geometry. The second section 106 includes two legs 106a, 106b that emerge from the braided stent body and cross over, or under, each other prior to traversing the circumference to connect to either end of the first section 100. Legs 106a, 106b are interconnected or woven into two or more closed-end loops. Stents 88, 90 advantageously permit a practitioner to directly grab the twisted portion 94, 102 or thread a device through the triangular portions 108, 110 below the twisted portions 94, 102, respectively. Threading a device through the triangular portions 108, 110 will automatically thread the device through the twisted portions 94, 102, once a pulling force is applied to the device to remove the device from the lumen and the stent. The pretzel formation thus provides a variety of ways to latch onto the stents 88, 90.

The retrieval and/or repositioning members of the present invention may be formed by wrapping one wire around template pins fixedly or removably disposed on a mandrel prior to winding or braiding the stent to form the first section and second section. The first section can be formed by wrapping the wire around a template pins positioned on the mandrel to cause the desired looped shape. The first section is a larger exaggerated section, such as grabbing area, for easy grabbing by the practitioner or physician. The first section may be bent or twisted as desired to form the desired geometric shape of the first section. The first section may be angularly bent and extended from the mandrel, the second section may extend from the angularly bend from the first section and perpendicularly from the first section. The second section may be formed by continuing to wrap the wire perimetrically about the mandrel forming the circumference of one end of the stent which is generally circular. A pulling force on the retrieval and/or repositioning member will cause cinching of the braid to a smaller diameter as it lengthens axially, thus allowing for less frictional force against the vessel wall and permitting retrieval and/or repositioning of the deployed stent. The retrieval and/or repositioning member wire is used to form the braided stent using the braiding technique as described herein. Additional details for braiding wires may be found in U.S. Application No. 61/147,307, filed Jan. 26, 2009, the contents of which are incorporated herein by reference.

The retrieval and/or repositioning member can be interlaced with one or more adjacent end loops as the braided stent is being formed. Having the retrieval and/or repositioning member interlaced with one or more, and desirably at least two, adjacent closed-end loops provides for cinching of the stent end upon applying the pulling force to the retrieval and/or repositioning member.

The stent of the present invention is made from a continuous single wire strand or a multiple of single wire strands. Further, a strand may include many wires that have been welded or attached together to form the continuous single strand. For example, multiple wires may be attached end to end to form a single continuous wire without edges and free unattached ends. Once the braiding of the stent has been completed, the ends of the wire, the beginning end and the ending end, may be connected together by various means, e.g., via welding, to form a continuous closed loop braided stent. Additionally, the retrieval and/or repositioning member may also have the same or different properties than other wire(s) which form the braided stent. For example, it may be of the same or different stiffness or flexibility, all of which may be tailored for a particular application. The choice of material, wire diameter, geometry and pre-treatment of the wires and stent configuration are some of the factors which may be varied to achieve particular stent properties. Additionally, as mentioned herein, the at least one retrieval and/or repositioning member may also be made radiopaque by various methods, for example with a coating or finish, with a band or as part of the stent material, as further described herein. Color or different finishes may also be added to the retrieval and/or repositioning member to visually differentiate it from the rest of the stent wires. In some embodiments such as were polymer wires are used, attachment means may include melting the polymeric wires

The stent may have weld joints which, due to their positioning, provide higher radial strength, i.e., the resultant stents can withstand higher radial compressive forces without fear of weld failure. Wire ends to be welded may be disposed about islands or gaps on a mandrel (not shown). After the welds are formed or while the welds are being formed wire portions not forming the stent may be cut or otherwise removed from the stent braiding pattern.

Further, the stent of the present invention may have a coating. In one embodiment, the coating is a tubular covering of silicone. The stent may be placed on a coating mandrel (not shown) and may further include a tie after which the assembly can be dipped into a silicone solution to form the coating. In one embodiment, the retrieval and/or repositioning member portion is not silicone covered. In one embodiment the coating or covering may be a silicone covering, but other coverings, particularly elastomeric polymers, are useful. The coating embeds the stent therein and essentially forms a stent covering. In some embodiments when coating, it may be desirable not to embed the retrieval and/or repositioning member section in the covering, although the other wire portions emanating from the retrieval and/or repositioning member which form the braid of the stent may be coated. To prevent coating of the retrieval and/or repositioning member section, the mandrel may be truncated or geometrically altered such that it does not permit coating of the retrieval and/or repositioning member, or the retrieval and/or repositioning member can be pulled away from the mandrel during coating and formation of the polymer covering.

The stent may be fully, substantially or partially covered or lined with a polymeric material. The stent may also be embedded in a polymeric coating. The covering may be in the form of a tubular structure. Nonlimiting examples of useful polymeric materials include polyesters, polypropylenes, polyethylenes, polyurethanes, polynaphthalenes, polytetrafluoroethylenes, expanded polytetrafluoroethylene, silicone, and combinations and copolymers thereof. In some embodiments, the polymeric material is silicone. The polymeric material and/or silicone may be disposed on external surfaces of the stent, or disposed on the internal surfaces of the stent or combinations thereof.

With any embodiment, the stent may be used for a number of purposes including to maintain patency of a body lumen, vessel or conduit, such as in the coronary or peripheral vasculature, esophagus, trachea, bronchi, colon, biliary tract, pancreatic duct, urinary tract, prostate, brain, and the like. The devices of the present invention may also be used to support a weakened body lumen or to provide a fluid-tight conduit for a body lumen.

Stents of the present invention, for example stent 30, may be placed at a variety of bodily locations. In some aspects of the present invention, the tubular wall 36 of the stent 30 is disposed with a bodily lumen and one end of the stent, for example stent end 32 with the retrieval and/or repositioning member 42, may be disposed beyond the bodily lumen being supported by the tubular wall 36 of the stent 30. In such cases, the retrieval and/or repositioning member 42 is often disposed in a larger bodily lumen organ such that the member 42 may be more easily accessed by a practitioner. For example, the tubular wall 36 of the stent 30 may be placed within the biliary duct and the stent end 32 with the retrieval and/or repositioning member 42 may be located within the duodenum where the member 42 is more easily accessed by a practitioner. Such aspects, however, are not limiting and the stent 30 may be suitably placed with any bodily lumen and/or organ including combinations of bodily lumens and/or organs.

The stent of the present invention may be treated with a therapeutic agent or agents. “Therapeutic agents”, “pharmaceuticals,” “pharmaceutically active agents”, “drugs” “genetic materials”, “biologically active materials” and other related terms may be used interchangeably herein and include genetic therapeutic agents, non-genetic therapeutic agents and cells. The term “genetic material” means DNA or RNA, including, without limitation, DNA/RNA encoding a useful protein stated below, intended to be inserted into a human body including viral vectors and non-viral vectors. Therapeutic agents may be used singly or in combination. A wide variety of therapeutic agents can be employed in conjunction with the present invention including those used for the treatment of a wide variety of diseases and conditions (i.e., the prevention of a disease or condition, the reduction or elimination of symptoms associated with a disease or condition, or the substantial or complete elimination of a disease or condition).

The term “biological materials” include cells, yeasts, bacterial, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (P0-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferring, cytotactin, cell binding domains (e.g., RGD), and tenascin. Exemplary BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Non-limiting examples of useful therapeutic agents include, but are not limited to, adrenergic agents, adrenocortical steroids, adrenocortical suppressants, alcohol deterrents, aldosterone antagonists, amino acids and proteins, ammonia detoxicants, anabolic agents, analeptic agents, analgesic agents, androgenic agents, anesthetic agents, anorectic compounds, anorexic agents, antagonists, anterior pituitary activators and suppressants, anthelmintic agents, anti-adrenergic agents, anti-allergic agents, anti-amebic agents, anti-androgen agents, anti-anemic agents, anti-anginal agents, anti-anxiety agents, anti-arthritic agents, anti-asthmatic agents, anti-atherosclerotic agents, antibacterial agents, anticholelithic agents, anticholelithogenic agents, anticholinergic agents, anticoagulants, anticoccidal agents, anticonvulsants, antidepressants, antidiabetic agents, antidiuretics, antidotes, antidyskinetics agents, anti-emetic agents, anti-epileptic agents, anti-estrogen agents, antifibrinolytic agents, antifungal agents, antiglaucoma agents, antihemophilic agents, antihemophilic Factor, antihemorrhagic agents, antihistaminic agents, antihyperlipidemic agents, antihyperlipoproteinemic agents, antihypertensives, antihypotensives, anti-infective agents, anti-inflammatory agents, antikeratinizing agents, antimicrobial agents, antimigraine agents, antimitotic agents, antimycotic agents, antineoplastic agents, anti-cancer supplementary potentiating agents, antineutropenic agents, antiobsessional agents, antiparasitic agents, antiparkinsonian drugs, antipneumocystic agents, antiproliferative agents, antiprostatic hypertrophydrugs, antiprotozoal agents, antipruritics, antipsoriatic agents, antipsychotics, antirheumatic agents, antischistosomal agents, antiseborrheic agents, antispasmodic agents, antithrombotic agents, antitussive agents, anti-ulcerative agents, anti-urolithic agents, antiviral agents, benign prostatic hyperplasia therapy agents, blood glucose regulators, bone resorption inhibitors, bronchodilators, carbonic anhydrase inhibitors, cardiac depressants, cardioprotectants, cardiotonic agents, cardiovascular agents, choleretic agents, cholinergic agents, cholinergic agonists, cholinesterase deactivators, coccidiostat agents, cognition adjuvants and cognition enhancers, depressants, diagnostic aids, diuretics, dopaminergic agents, ectoparasiticides, emetic agents, enzyme inhibitors, estrogens, fibrinolytic agents, free oxygen radical scavengers, gastrointestinal motility agents, glucocorticoids, gonad-stimulating principles, hemostatic agents, histamine H2 receptor antagonists, hormones, hypocholesterolemic agents, hypoglycemic agents, hypolipidemic agents, hypotensive agents, HMGCoA reductase inhibitors, immunizing agents, immunomodulators, immunoregulators, immunostimulants, immunosuppressants, impotence therapy adjuncts, keratolytic agents, LHRH agonists, luteolysin agents, mucolytics, mucosal protective agents, mydriatic agents, nasal decongestants, neuroleptic agents, neuromuscular blocking agents, neuroprotective agents, NMDA antagonists, non-hormonal sterol derivatives, oxytocic agents, plasminogen activators, platelet activating factor antagonists, platelet aggregation inhibitors, post-stroke and post-head trauma treatments, progestins, prostaglandins, prostate growth inhibitors, prothyrotropin agents, psychotropic agents, radioactive agents, repartitioning agents, scabicides, sclerosing agents, sedatives, sedative-hypnotic agents, selective adenosine A1 antagonists, adenosine A2 receptor antagonists (e.g., CGS 21680, regadenoson, UK 432097 or GW 328267), serotonin antagonists, serotonin inhibitors, serotonin receptor antagonists, steroids, stimulants, thyroid hormones, thyroid inhibitors, thyromimetic agents, tranquilizers, unstable angina agents, uricosuric agents, vasoconstrictors, vasodilators, vulnerary agents, wound healing agents, xanthine oxidase inhibitors, and the like, and combinations thereof.

Useful non-genetic therapeutic agents for use in connection with the present invention include, but are not limited to,
(a) anti-thrombotic agents such as heparin, heparin derivatives, urokinase, clopidogrel, and PPack (dextrophenylalanine proline arginine chloromethylketone);
(b) anti-inflammatory agents such as glucorticoids, betemethasone, dexamethasone, prednisolone, corticosterone, budesonide, estrogen, sulfasalazine and mesalamine;
(c) antineoplastic/antiproliferative/anti-miotic agents such as paclitaxel, 5-fluorouracil, cisplatin, vinblastine, vincristine, epothilones, methotrexate, azathioprine, adriamycin and mutamycin, endostatin, angiostatin, angiopeptin, monoclonal antibodies capable of blocking smooth muscle cell proliferation, thymidine kinase inhibitors, cladribine, taxol and its analogs or derivatives, paclitaxel as well as its derivatives, analogs or paclitaxel bound to proteins, e.g. Abraxane™;
(d) anesthetic agents such as lidocaine, bupivacaine and ropivacaine;
(e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
(f) vascular cell growth promoters such as growth factors, vascular endothelial growth factors (VEGF, all types including VEGF-2), growth factor receptors, transcriptional activators, and translational promotors;
(g) vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
(h) protein kinase and tyrosine kinase inhibitors (e.g., tyrphostins, genistein, quinoxalines);
(i) prostacyclin analogs;
(j) cholesterol-lowering agents;
(k) angiopoietins;
(l) antimicrobial agents such as triclosan, cephalosporins, aminoglycosides and nitrofurantoin;
(m) cytotoxic agents, cytostatic agents and cell proliferation affectors;
(n) vasodilating agents;
(o) agents that interfere with endogenous vasoactive mechanisms;
(p) inhibitors of leukocyte recruitment, such as monoclonal antibodies;
(q) cytokines;
(r) hormones;
(s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin;
(t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, diclomine);
(u) bARKct inhibitors;
(v) phospholamban inhibitors;
(w) Serca 2 gene/protein;
(x) immune response modifiers including aminoquizolines, for instance, imidazoquinolines such as resiquimod and imiquimod;
(y) human apolioproteins (e.g., AI, AII, AIII, AIV, AV, etc.);
(z) selective estrogen receptor modulators (SERMs) such as raloxifene, lasofoxifene, arzoxifene, miproxifene, ospemifene, PKS 3741, MF 101 and SR 16234;
(aa) PPAR agonists, including PPAR-alpha, gamma and delta agonists, such as rosiglitazone, pioglitazone, netoglitazone, fenofibrate, bexaotene, metaglidasen, rivoglitazone and tesaglitazar;
(bb) prostaglandin E agonists, including PGE2 agonists, such as alprostadil or ONO 8815Ly;
(cc) thrombin receptor activating peptide (TRAP);
(dd) vasopeptidase inhibitors including benazepril, fosinopril, lisinopril, quinapril, ramipril, imidapril, delapril, moexipril and spirapril;
(ee) thymosin beta 4;
(ff) phospholipids including phosphorylcholine, phosphatidylinositol and phosphatidylcholine;
(gg) VLA-4 antagonists and VCAM-1 antagonists;
(hh) anti-proliferative agents such as enoxaprin, angiopeptin, or monoclonal antibodies capable of blocking smooth muscle cell proliferation, hirudin, acetylsalicylic acid, tacrolimus, everolimus, pimecrolimus, sirolimus, zotarolimus, amlodipine and doxazosin;
(ii) DNA demethylating drugs such as 5-azacytidine, which is also categorized as a RNA or DNA metabolite that inhibit cell growth and induce apoptosis in certain cancer cells;
(jj) cholesterol-lowering agents, vasodilating agents, and agents which interfere with endogenous vasoactive mechanisms;
(kk) anti-oxidants, such as probucol;
(ll) antibiotic agents, such as penicillin, cefoxitin, oxacillin, tobranycin, erythromycin, amphotericin, rapamycin (sirolimus) and adriamycin;
(mm) angiogenic substances, such as acidic and basic fibroblast growth factors, estrogen including estradiol (E2), estriol (E3) and 17-beta estradiol;
(nn) drugs for heart failure, such as digoxin, beta-blockers, angiotensin-convertin enzyme (ACE) inhibitors including captropril and enalopril, statins and related compounds; and
(oo) macrolides such as sirolimus or everolimus.
   The non-genetic therapeutic agents may be used individually or in combination, including in combination with any of the agents described herein.

Other therapeutic agents include nitroglycerin, nitrous oxides, nitric oxides, antibiotics, aspirins, digitalis, estrogen, estradiol, halafuginone, phospholamban inhibitors and glycosides. Exemplary therapeutic agents include anti-proliferative drugs such as steroids, vitamins, and restenosis-inhibiting agents. Exemplary restonosis-inhibiting agents include microtubule stabilizing agents such as Taxol®, paclitaxel (i.e., paclitaxel, paxlitaxel analogs, or paclitaxel derivatives, and mixtures thereof). For example, derivatives suitable for use in the medical devices include 2′-succinyl-taxol, 2′-succinyl-taxol triethanolamine, 2′-glutaryl-taxol, 2′ glutaryl-taxol triethanolamine salt, 2′-O-ester with N-(dimethylaminoethyl)glutamine, and 2′-O-ester with N-(dimethylaminoethyl)glutamide hydrochloride salt.

Further examples of non-genetic therapeutic agents, not necessarily exclusive of those listed above, include taxanes such as paclitaxel (including particulate forms thereof, for instance, protein-bound paclitaxel particles such as albumin-bound paclitaxel nanoparticles, e.g., Abraxane™), sirolimus, everolimus, tacrolimus, zotarolimus, Epo D, dexamethasone, estradiol, halofuginone, cilostazole, geldanamycin, alagebrium chloride (ALT-711), ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well derivatives of the forgoing, among others.

Useful genetic therapeutic agents for use in connection with the present invention include, but are not limited to, anti-sense DNA and RNA as well as DNA coding for the various proteins (as well as the proteins themselves), such as (a) anti-sense RNA; (b) tRNA or rRNA to replace defective or deficient endogenous molecules; (c) angiogenic and other factors including growth factors such as acidic and basic fibroblast growth factors, vascular endothelial growth factor, endothelial mitogenic growth factors, epidermal growth factor, transforming growth factor α and β, platelet-derived endothelial growth factor, platelet-derived growth factor, tumor necrosis factor α, hepatocyte growth factor and insulin-like growth factor; (d) cell cycle inhibitors including CD inhibitors, and (e) thymidine kinase (“TK”) and other agents useful for interfering with cell proliferation. DNA encoding for the family of bone morphogenic proteins (“BMP's”) are also useful and include, but not limited to, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently desirably BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include, but not limited to, viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers such as polyvinylpyrrolidone (PVP), SP1017 (SUPRATEK), lipids such as cationic lipids, liposomes, lipoplexes, nanoparticles, or microparticles, with and without targeting sequences such as the protein transduction domain (PTD).

Cells for use in connection with the present invention may include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Numerous therapeutic agents, not necessarily exclusive of those listed above, have been identified as candidates for vascular treatment regimens, for example, as agents targeting restenosis (antirestenotics). Such agents are useful for the practice of the present invention and include one or more of the following:
(a) Ca-channel blockers including benzothiazapines such as diltiazem and clentiazem, dihydropyridines such as nifedipine, amlodipine and nicardapine, and phenylalkylamines such as verapamil;
(b) serotonin pathway modulators including: 5-HT antagonists such as ketanserin and naftidrofuryl, as well as 5-HT uptake inhibitors such as fluoxetine;
(c) cyclic nucleotide pathway agents including phosphodiesterase inhibitors such as cilostazole and dipyridamole, adenylate/Guanylate cyclase stimulants such as forskolin, as well as adenosine analogs;
(d) catecholamine modulators including α-antagonists such as prazosin and bunazosine, β-antagonists such as propranolol and α/β-antagonists such as labetalol and carvedilol;
(e) endothelin receptor antagonists such as bosentan, sitaxsentan sodium, atrasentan, endonentan;
(f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine;
(g) Angiotensin Converting Enzyme (ACE) inhibitors such as cilazapril, fosinopril and enalapril;
(h) ATII-receptor antagonists such as saralasin and losartin;
(i) platelet adhesion inhibitors such as albumin and polyethylene oxide;
(j) platelet aggregation inhibitors including cilostazole, aspirin and thienopyridine (ticlopidine, clopidogrel) and GP IIb/IIIa inhibitors such as abciximab, epitifibatide and tirofiban;
(k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C;
(l) cyclooxygenase pathway inhibitors such as aspirin, ibuprofen, flurbiprofen, indomethacin and sulfinpyrazone;
(m) natural and synthetic corticosteroids such as dexamethasone, prednisolone, methprednisolone and hydrocortisone;
(n) lipoxygenase pathway inhibitors such as nordihydroguairetic acid and caffeic acid;
(o) leukotriene receptor antagonists; (p) antagonists of E- and P-selectins;
(q) inhibitors of VCAM-1 and ICAM-1 interactions;
(r) prostaglandins and analogs thereof including prostaglandins such as PGE1 and PGI2 and prostacyclin analogs such as ciprostene, epoprostenol, carbacyclin, iloprost and beraprost;
(s) macrophage activation preventers including bisphosphonates;
(t) HMG-CoA reductase inhibitors such as lovastatin, pravastatin, atorvastatin, fluvastatin, simvastatin and cerivastatin;
(u) fish oils and omega-3-fatty acids;
(v) free-radical scavengers/antioxidants such as probucol, vitamins C and E, ebselen, trans-retinoic acid, SOD (orgotein) and SOD mimics, verteporfin, rostaporfin, AGI 1067, and M 40419;
(w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives;
(x) matrix metalloprotease (MMP) pathway inhibitors such as marimastat, ilomastat, metastat, batimastat, pentosan polysulfate, rebimastat, incyclinide, apratastat, PG 116800, RO 1130830 or ABT 518;
(y) cell motility inhibitors such as cytochalasin B;
(z) antiproliferative/antineoplastic agents including antimetabolites such as purine antagonists/analogs (e.g., 6-mercaptopurine and pro-drugs of 6-mercaptopurine such as azathioprine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), olimus family drugs (e.g., sirolimus, everolimus, tacrolimus, zotarolimus, etc.), cerivastatin, flavopiridol and suramin;
(aa) matrix deposition/organization pathway inhibitors such as halofuginone or other quinazolinone derivatives, pirfenidone and tranilast;
(bb) endothelialization facilitators such as VEGF and RGD peptide;
(cc) blood rheology modulators such as pentoxifylline and
(dd) glucose cross-link breakers such as alagebrium chloride (ALT-711).
   These therapeutic agents may be used individually or in combination, including in combination with any of the agents described herein.

Numerous additional therapeutic agents useful for the practice of the present invention are also disclosed in U.S. Pat. No. 5,733,925 to Kunz, the contents of which is incorporated herein by reference.

A wide range of therapeutic agent loadings may used in connection with the dosage forms of the present invention, with the pharmaceutically effective amount being readily determined by those of ordinary skill in the art and ultimately depending, for example, upon the condition to be treated, the nature of the therapeutic agent itself, the tissue into which the dosage form is introduced, and so forth.

Further, with any embodiment of the stent the general tubular shape may be varied. For example, the tubular shape may have a varied diameter, may be tapered, and may have an outwardly flared end and the like. Further, the ends of the stent may have a larger diameter than the middle regions of the stent. In one particularly useful embodiment, at least one of the ends of the stent transition from one diameter to another diameter. Desirably, both ends transition in this manner to yield “flared” ends.

The stent may be coated with a polymeric material. For example, the stent wires may be partially or fully covered with a biologically active material which is elutably disposed with the polymeric material. Further, the polymeric coating may extend over or through the interstitial spaces between the stent wires so as to provide a hollow tubular liner or cover over the interior or the exterior surface of the stent. The polymeric material may be selected from the group consisting of polyester, polypropylene, polyethylene, polyurethane, polynaphthalene, polytetrafluoroethylene, expanded polytetrafluoroethylene, silicone, and combinations thereof.

Various stent types and stent constructions may be employed in the invention. Among the various stents useful include, without limitation, self-expanding stents and balloon expandable extents. The stents may be capable of radially contracting, as well and in this sense can best be described as radially distensible or deformable. Self-expanding stents include those that have a spring-like action which causes the stent to radially expand, or stents which expand due to the memory properties of the stent material for a particular configuration at a certain temperature. Nitinol is one material which has the ability to perform well while both in spring-like mode, as well as in a memory mode based on temperature. Other materials are of course contemplated, such as stainless steel, platinum, gold, titanium and other biocompatible metals, as well as polymeric stents. The configuration of the stent may also be chosen from a host of geometries. For example, wire stents can be fastened into a continuous helical pattern, with or without a wave-like or zig-zag in the wire, to form a radially deformable stent. Individual rings or circular members can be linked together such as by struts, sutures, welding or interlacing or locking of the rings to form a tubular stent. Tubular stents useful in the present invention also include those formed by etching or cutting a pattern from a tube. Such stents are often referred to as slotted stents. Furthermore, stents may be formed by etching a pattern into a material or mold and depositing stent material in the pattern, such as by chemical vapor deposition or the like. Examples of various stent configurations are shown in U.S. Pat. No. 4,503,569 to Dotter; U.S. Pat. No. 4,733,665 to Palmaz; U.S. Pat. No. 4,856,561 to Hillstead; U.S. Pat. No. 4,580,568 to Gianturco; U.S. Pat. No. 4,732,152 to Wallsten, U.S. Pat. No. 4,886,062 to Wiktor, and U.S. Pat. No. 5,876,448 to Thompson, U.S. Pat. Nos. 6,007,574, 6,309,415, 7,60,323, 7,419,502 and 7,419,503 to Pulnev et al.; U.S. Pat. No. 7,311,031 to McCullagh et al.; and U.S. Pat. Application Publication No. 2007/0118206 to Colgan et al., all of whose contents are incorporated herein by reference.

The invention being thus described, it will now be evident to those skilled in the art that the same may be varied in many ways. Such variations are not to be regarded as a departure from the spirit and scope of the invention and all such modifications are intended to be included within the scope of the following claims. Further, any of the embodiments or aspects of the invention as described in the claims may be used with one and another without limitation.

What is claimed is:

1. An implantable device comprising:
one or more elongated wires braided to form a tubular device having opposed first open end, a second open end, a tubular body therebetween defining a device lumen therethrough with an interior surface and an exterior surface, and a retrieval and/or repositioning member integrally formed from a single of said elongated wires, said retrieval and/or repositioning member includes an elongated portion extending from said first open end and said retrieval and/or repositioning member interlooping circumferentially about said first open end whereby force exerted on said elongated portion causes radially contraction of said tubular stent and cinching of said first open end.

2. The device of claim 1, wherein said first open end is defined by a series of closed-end loops and said retrieval and/or repositioning member passes through at least two closed-end loops attaching said elongated portion to said first open end.

3. The device of claim 1, wherein said retrieval and/or repositioning member consists essentially of said single wire which extends about said first open end and crosses over itself prior to being incorporated into said tubular body.

4. The device of claim 1, wherein said elongated portion of said retrieval and/or repositioning member is a single wire loop.

5. The device of claim 4, wherein said single wire loop is bent inwardly towards said stent lumen forming an inwardly directed bent portion.

6. The device of claim 4, wherein said single wire loop is bent outwardly away from said device lumen forming an externally directed bent portion.

7. The device of claim 4, wherein said inwardly directed bent portion is twisted into a knot.

8. The device of claim 1, wherein said retrieval and/or repositioning member includes two elongated portions extending from said first open end forming two single wire loops.

9. The device of claim 8, wherein each of said two elongated portions are loops bent into a hook shaped geometry directed outwardly away from said device lumen.

10. The device of claim 8, wherein each of said two elongated portions are loops bent into a hook shaped geometry directed inwardly toward said device lumen.

11. The device of claim 1, wherein said wire comprises metallic and/or polymeric materials.

12. The device of claim 1, further comprising a covering disposed over at least a portion of a device surface.

13. A stent comprising:
one or more elongated wires braided to form a tubular stent having a retrieval and/or repositioning member and opposed first open end and a second open end with each open end having a circumference and a tubular body therebetween, said first open end is defined by series of closed-end loops, said retrieval and/or repositioning member having a first section including at least one elongated closed-end loop extending from said first open end and a second section emerging from said braided tubular body, interwoven with at least one closed-end loop and integrally extending into said first section whereby force exerted on said elongated closed-end loop causes radially contraction of said tubular stent.

14. A method for producing a tubular braided stent having opposed first stent end and second stent end and having an integral retrieval and/or repositioning loop at the first stent end, comprising:
selecting one or more elongate wires having opposed ends;
forming a retrieval and/or repositioning member from single of said wires comprising an elongated loop which extends above and beyond said first stent end to permit grabbing of said loop by a practitioner to radially contract said stent; and
braiding said one or more wires to form said stent.

15. The method of claim 14, wherein the step for braiding includes interweaving said retrieval and/or repositioning loop circumferentially into said first stent end.

16. The method of claim 15, further comprising bending said elongated loop to form a bent hook portion extending outwardly from said stent.

17. The method of claim 154, further comprising bending said elongated loop to form a bent hook portion extending inwardly towards said stent end.

18. The method of claim 17, further comprising twisting said bent hook portion to form a twisted knot shaped portion.

19. The method of claim 14, wherein said step of forming a retrieval and/or repositioning member includes forming two opposing elongated loops which extend above and beyond said first stent end to permit grabbing of said loops by a practitioner to radially contract said stent.

20. A delivery system comprising:
a delivery catheter; and
a stent comprising: one or more elongated wires braided to form a tubular stent having opposed first open end, a second open end, a tubular body therebetween defining a stent lumen therethrough with an interior surface and an exterior surface, and a retrieval and/or repositioning member integrally formed from a single of said elongated wires, said retrieval and/or repositioning member includes an elongated portion extending from said first open end and said retrieval and/or repositioning member interwoven circumferentially about said first open end whereby force exerted on said elongated portion causes radially contraction of said tubular stent and cinching of said first open end.
